(19) United States
(12) Patent Application Publication
(10) Pub. No.: US 2016/0045439 A1
(43) Pub. Date: Feb. 18, 2016

(54) COMPOSITIONS FOR INHIBITING INFLAMMATION IN A SUBJECT WITH A SPINAL CORD INJURY AND METHODS OF USING THE SAME

(71) Applicant: PixarBio Corporation, Medford, MA (US)

(72) Inventors: JASON M. CRISCIONE, CHELMSFORD, MA (US); FRANCIS M. REYNOLDS, SALEM, NH (US); HAINING DAI, ROCKVILLE, MD (US)

(21) Appl. No.: 14/826,541

(22) Filed: Aug. 14, 2015

Related U.S. Application Data
(60) Provisional application No. 62/037,628, filed on Aug. 15, 2014.

(51) Int. Cl. A61K 9/16; C07K 16/24; A61K 39/395; A61K 47/48; C08G 63/91; A61K 9/00; A61K 31/416; A61K 31/192; A61K 31/65; A61K 31/4418; A61K 9/06

(52) U.S. Cl. CPC A61K 9/1647; A61K 31/4418; C07K 16/241; A61K 39/3955; A61K 47/48915; A61K 9/06; A61K 9/0085; A61K 31/416; A61K 31/192; A61K 31/65; A61K 9/0019; C07K 16/24; C08G 63/912; C07K 2317/76

(57) ABSTRACT

Provided herein are compositions for inhibiting inflammation in a subject with a spinal cord injury comprising one or more agents capable of specifically reducing TNF-α signaling and a biodegradable carrier. Further provided herein are compositions for inhibiting inflammation in a subject with a spinal cord injury comprising one or more agents capable of modulating MCP-1 signaling and a biodegradable carrier. Methods of treating inflammation in a subject having a spinal cord injury and kits for producing the compositions are also disclosed.

### CROSS-REFERENCE TO RELATED APPLICATIONS

The present application claims priority to U.S. Provisional Application No. 62/037,628, filed Aug. 15, 2014, the entire contents of which are incorporated herein by reference.

### TECHNICAL FIELD

Provided herein are compositions, methods, and kits for inhibiting inflammation in a subject with a spinal cord injury.

### BACKGROUND

Spinal cord injury (SCI) affect tens of thousands of people annually worldwide and over 12,000 people annually in the United States of America. In the days to weeks following primary injury, secondary injury processes advance to increase the severity of the SCI resulting in additional structure and function loss due to complications, such as inflammation and oxidative stress. The medical community has not yet found an effective treatment to reduce the inflammation and neuroprotect the patient's spinal cord tissue, leaving patients with significant long-term disability. Many studies have found the inflammatory process, specifically, monocyte and macrophage recruitment to and infiltration of the lesion region, to play a crucial role in the occurrence and progression of secondary injury [Ren et al., Neural Plasticity., 2013, 2013:945034; Gensel et al., Brain Research., 2015, 1619: 1-11]. During the progression of the secondary injury, the cytokine and chemokine milieu dictates the subsets of recruited and activated macrophages [Oyinbo, Acta Neurobiol Exp., 2011, 71: 281-299; Lee et al., Neurochem Int., 2000, 36: 417-425]. For example, TNF-α regulated JNK-induced secretion of the chemokine, MCP-1, represents a dominant pathway for initiating the recruitment of monocytes and macrophage to the injury site [Gao et al., J Neuroscience., 2009, 29(13): 4096-4108; Lee et al., Neurochem Int., 2000, 36: 417-425; Perrin et al., Brain., 2005, 128: 854-866;]. Pro-inflammatory or Th1 cytokines (e.g. TNF-α, IL-1β) skew macrophage activation to the classical M1 phenotype. The M1 phenotype is responsible for generating tissue inflammation, demyelination, and degeneration [Ren et al., Neural Plasticity., 2013, 2013:945034; Gensel et al., Brain Research., 2015, 1619: 1-11]. In contrast, anti-inflammatory or Th2 cytokines (e.g. IL-10, IL-4, TGF-β) skew macrophage activation to the M2 phenotype. The M2 phenotype is responsible for generating wound healing and tissue remodeling [Ren et al., Neural Plasticity., 2013, 2013:945034; Gensel et al., Brain Research., 2015, 1619: 1-11]. The severity of the secondary injury is potentiated by the persistence of M1 macrophages at the injury site, as this extends the inflammatory response and inhibits the proper initiation of remodeling and regeneration.

Though immune-modulation is often a “double-edged sword”, in the case of secondary injury after SCI, immunotherapeutic approaches designed to skew the local microenvironment away from a Th1 response and towards a Th2 response represent an attractive means to reduce inflammation and improve functional recovery. Recently, targeting inhibition of pro-inflammatory cytokines and chemokines (e.g. TNF-α and MCP-1, respectively) have demonstrated potential as treatment strategies for SCI [Ren et al., Neural Plasticity., 2013, 2013:945034; Esposito et al., Trends Pharmacol Sci., 2011, 32(2) 107-115]. For example, blockade of TNF-α with TNF-α inhibiting antibodies (e.g. infliximab, etanercept) has been observed to improve functional recovery after SCI. While these immunotherapeutic approaches show promise as treatment strategies for SCI, systemic delivery of TNF-α inhibitors has associated risks and undesired, pleiotropic side effects. Consequently, physicians cannot always dose enough drug to have the desired anti-inflammatory effect without causing problematic, pleiotropic systemic side effects. Local delivery of the disclosed immunotherapeutic agents would abrogate these pleiotropic, systemic side effects and enable their therapeutic intervention for the management of secondary injury after SCI. For example, a localized injection of a depot formulation of a TNF-α inhibiting agent would permit the use of a lower initial dose than would be required for systemic or oral administration of the agent because the depot would establish therapeutically efficacious concentrations of the agent specifically at the desired site of action.

Recently, along these lines, biodegradable nanoparticles have been explored as a means to achieve local delivery to promote the inhibition of astrocyte growth in the treatment of SCI [Ren et al., Biomaterials., 2014, 35: 6585-6594]. Specifically, inhibition of astrocyte growth in a hemi-section rodent model of SCI through the local delivery of PLGA nanoparticles incorporating the cell-cycle inhibitor, flavopiridol, resulted in improved functional recovery after SCI.

Although there are still many unknowns about such treatments, many are hopeful that immunotherapeutic approaches designed to modulate the inflammatory process to enable neuroprotection can limit the advancement of the secondary injury, thereby reducing the severity of a spinal cord injury. Further, approaches designed to locally deliver these immunotherapeutics directly to the site on injury will enable abrogation of undesired, pleiotropic side effects, thus extending their utility in the treatment of SCI.

The disclosed compositions, methods, and kits address these and other important needs.

### SUMMARY

Provided herein are compositions for inhibiting inflammation in a subject with a spinal cord injury comprising one or more agents capable of specifically reducing TNF-α signaling and a biodegradable carrier.

Also provided herein are compositions for inhibiting inflammation in a subject with a spinal cord injury comprising one or more agents capable of modulating MCP-1 signaling and a biodegradable carrier.

Methods of treating inflammation in a subject having a spinal cord injury comprising administering the disclosed compositions and kits for producing the disclosed compositions are also provided.

### BRIEF DESCRIPTION OF THE DRAWINGS

The summary, as well as the following detailed description, is further understood when read in conjunction with the appended drawings. For the purpose of illustrating the invention, there are shown in the drawings exemplary embodiments of the invention; however, the invention is not limited to the specific compositions, methods, and kits disclosed. In the drawings:

FIG. 1, comprising FIGS. 1A-1B, represents an exemplary composition comprising A) one or more agents incorporated within a biodegradable carrier and B) release of the agent upon degradation of the carrier.

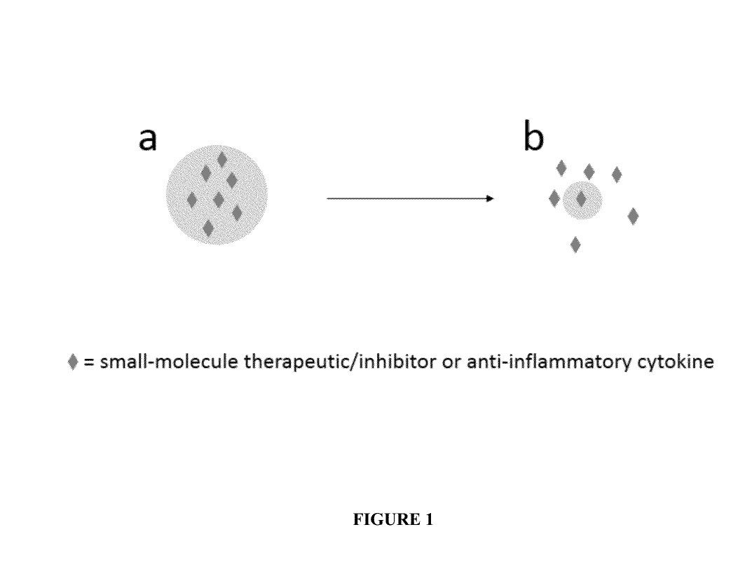

FIG. 2, comprising FIGS. 2A-2B, represents an exemplary composition comprising A) an agent, exposed on the surface of a biodegradable carrier, which is capable of specifically binding TNF-α or MCP-1 and B) binding of the agent to TNF-α or MCP-1.

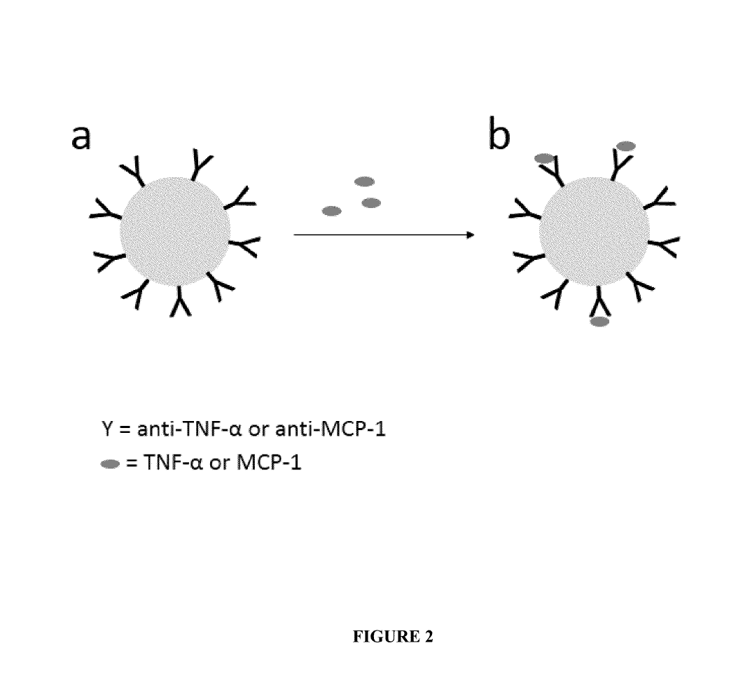

FIG. 3, comprising FIGS. 3A-3B, represents an exemplary composition comprising A) one or more agents exposed on the surface of the biodegradable carrier and one or more agents incorporated within the biodegradable carrier and B) the binding of the agent exposed on the surface to TNF-α or MCP-1 and the release of the incorporated agent.

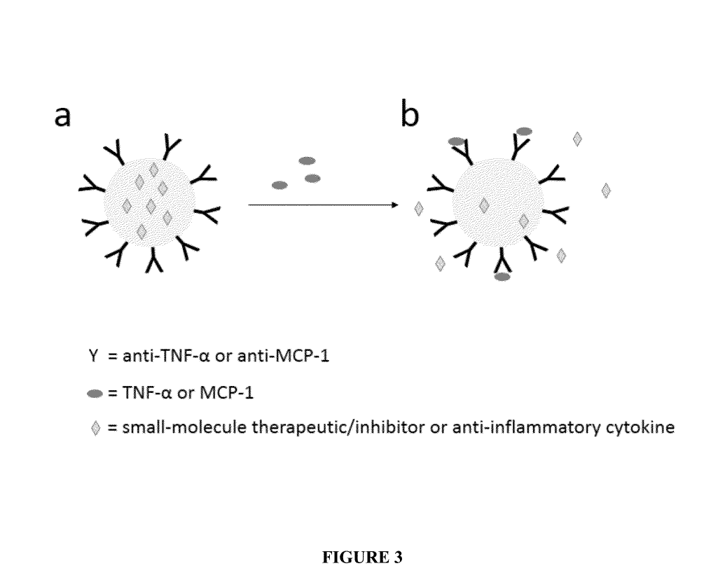

### DETAILED DESCRIPTION OF ILLUSTRATIVE EMBODIMENTS

The disclosed compositions, methods, and kits may be understood more readily by reference to the following detailed description taken in connection with the accompanying figures, which form a part of this disclosure. It is to be understood that the disclosed compositions, methods, and kits are not limited to the specific compositions, methods, and kits described and/or shown herein, and that the terminology used herein is for the purpose of describing particular embodiments by way of example only and is not intended to be limiting of the claimed compositions, methods, and kits. Also, as used in the specification including the appended claims, the singular forms “a,” “an,” and “the” include the plural, and reference to a particular numerical value includes at least that particular value, unless the context clearly dictates otherwise. When a range of values is expressed, another embodiment includes from the one particular value and/or to the other particular value. Further, reference to values stated in ranges include each and every value within that range. All ranges are inclusive and combinable. Similarly, when values are expressed as approximations, by use of the antecedent “about,” it will be understood that the particular value forms another embodiment.

It is to be appreciated that certain features of the disclosed compositions, methods, and kits which are, for clarity, described herein in the context of separate embodiments, may also be provided in combination in a single embodiment. Conversely, various features of the disclosed compositions, methods, and kits that are, for brevity, described in the context of a single embodiment, may also be provided separately or in any subcombination.

The term “about” when used in reference to numerical ranges, cutoffs, or specific values is used to indicate that the recited values may vary by up to as much as 25% from the listed value. As many of the numerical values used herein are experimentally determined, it should be understood by those skilled in the art that such determinations can, and often times will, vary among different experiments. The values used herein should not be considered unduly limiting by virtue of this inherent variation. The term “about” is used to encompass variations of ±25% or less, variations of ±20% or less, variations of 10% or less, variations of ±5% or less, variations of ±1% or less, variations of ±0.5% or less, or variations of ±0.1% or less from the specified value.

As used herein, “administering to said subject” and similar terms indicate a procedure by which one or more of the described agents or compositions, together or separately, are introduced into, implanted in, injected into, or applied onto a subject such that target cells, tissues, or segments of the body of the subject are contacted with the agent.

“Pharmaceutically acceptable” refers to those properties and substances which are acceptable to the patient from a pharmacological/toxicological point of view and to the manufacturing pharmaceutical chemist from a physical/chemical point of view regarding composition, formulation, stability, patient acceptance, and bioavailability.

“Pharmaceutically acceptable carrier” refers to a medium that does not interfere with the effectiveness of the biological activity of the active ingredient(s) and is not toxic to the host to which it is administered.

“Therapeutically effective dose” refers to an amount of a composition, as described herein, effective to achieve a particular biological or therapeutic result such as, but not limited to, biological or therapeutic results disclosed, described, or exemplified herein. The therapeutically effective dose may vary according to factors such as the disease state, age, sex, and weight of the individual, and the ability of the composition to cause a desired response in a subject. Such results may include, but are not limited to, the treatment of a spinal cord injury, as determined by any means suitable in the art.

The terms “treating” or “treatment” refer to any success or indicia of success in the attenuation or amelioration of an injury, pathology or condition, including any objective or subjective parameter such as abatement, remission, diminishing of symptoms or making the injury, pathology, or condition more tolerable to the patient, slowing in the rate of inflammation, making the final point of inflammation less debilitating, improving a subject's physical or mental well-being, or prolonging the length of survival. The treatment may be assessed by objective or subjective parameters; including the results of a physical examination, neurological examination, or psychiatric evaluations.

As used herein, the term “specifically” refers to the ability of a protein to bind to TNF-α or MCP-1 with higher selectivity and affinity than other proteins.

As used herein, “exposed on the surface” means that at least a portion of the one or more agents is not covered or encased by the biodegradable carrier and is accessible from the exterior of the biodegradable carrier. The one or more agents exposed on the surface can be fully exposed, such that the entire agent is on the surface of the biodegradable carrier, or can be partially exposed, such that only a portion of the agent is on the surface of the biodegradable carrier. The one or more agents that are exposed on the surface of the biodegradable carrier can be bound to the surface of the biodegradable carrier through, for example, covalent or non-covalent bonds, or can be incorporated within the biodegradable carrier such that a portion of the agent is exposed on the surface.

As used herein, “incorporated within” means that the one or more agents are at least partially covered by, contained within, encased in, or entrapped by the biodegradable carrier. In such circumstances, the one or more agents may or may not be exposed on the surface of the biodegradable carrier. Depending on the type of biodegradable carrier present in the composition, the one or more agents may be located in a void space, such as a core, of the biodegradable carrier or dispersed within the biodegradable carrier with the potential for being exposed on the surface, or any combination thereof. In some embodiments, the one or more agents can be dispersed or distributed within the biodegradable carrier, and not partially exposed on the surface of the biodegradable carrier. In other embodiments, the one or more agents can be partially exposed on the surface of the biodegradable carrier. In other embodiments, the one or more agents can be both dispersed or distributed within the biodegradable carrier and partially exposed on the surface of the biodegradable carrier. In yet other embodiments, the one or more agents can be located in a void space of the biodegradable carrier. In yet other embodiments, the one or more agents can be both located in a void space of the biodegradable carrier and exposed on the surface of the biodegradable carrier.

As used herein, “reduce TNF-α signaling” includes complete or partial inhibition of TNF-α signaling. Reduction of TNF-α signaling can be the result of, for example, sequestration of, and/or degradation of, TNF-α.

As used herein, “modulate MCP-1 signaling” means the complete or partial reduction of MCP-1 signaling, and includes direct and indirect modulation of MCP-1 signaling. For example, the one or more agents can bind directly to MCP-1 preventing MCP-1 from interacting with and/or activating its receptor. Alternatively, the one or more agents can indirectly modulate MCP-1 signaling by inhibiting other proteins or factors that function to produce or release MCP-1 or that are involved in MCP-1 signaling. Furthermore, the one or more agents can indirectly modulate MPC-1 signaling by activating proteins or factors that in turn inactivate MCP-1 signaling.

### Compositions Comprising One or More Agents Capable of Specifically Reducing TNF-α Signaling

Disclosed herein are compositions for inhibiting inflammation in a subject with a spinal cord injury comprising, one or more agents capable of specifically reducing TNF-α signaling, and a biodegradable carrier.

Suitable biodegradable carriers include, but are not limited to, a microparticle, a nanoparticle, a hydrogel, or any combination thereof.

Biodegradable carriers can comprise synthetically derived polymers, including, biodegradable polymers. Exemplary polymers include, but are not limited to, poly(lactides) (PLA), poly(glycolides) (PGA), poly(lactide-co-glycolides) (PLGA), poly(ethylene glycols)(PEG), or any combination thereof. In some embodiments, the synthetically derived biodegradable polymer can be poly(lactic-co-glycolic acid) (PLGA), having a lactic acid and glycolic acid content ranging from 0-100% for each monomer. For example, in some aspects, the biodegradable polymer can be a 50:50 PLGA, where 50:50 refers to the ratio of lactic to glycolic acid. In some embodiments, the biodegradable carrier comprises or consists of a copolymer. For example, in some embodiments, the biodegradable polymer can be a copolymer of poly(ethylene glycol) (PEG) and poly(lactic-co-glycolic acid) (PLGA), having a lactic acid and glycolic acid content ranging from 0-100% for each monomer. Further, in some embodiments, the biodegradable carrier can be a microparticle and/or nanoparticle comprising 50:50 PLGA. In other embodiments, the biodegradable carrier can be a microparticle and/or nanoparticle comprising a copolymer of 50:50 PLGA and PEG. In yet other embodiments, the biodegradable carrier can be a hydrogel comprising PEGs and/or copolymers of PEG and PLGA.

Exemplary biodegradable microparticles and/or nanoparticles can be fabricated using processing techniques known by those skilled in the art, including, but not limited to, emulsification, precipitation, or spray drying. In some embodiments, the microparticles and/or nanoparticles can be fabricated by emulsification. In other embodiment, the microparticles and/or nanoparticles can be fabricated by precipitation or nanoprecipitation, respectively. In yet other embodiments, the microparticles and/or nanoparticles can be fabricated by spray drying.

Exemplary biodegradable hydrogels can be designed to be injectable and capable of forming in situ by methods and crosslinking chemistries known by those skilled in the art, including, but not limited to, crosslinking by copper-free click chemistry, crosslinking by Michael-type addition, gelation by a shear-thinning mechanism, gelation by a thermosensitive mechanism, or any combination thereof. In some embodiments, the injectable hydrogel can be formed in situ by copper-free click chemistry crosslinking. In some embodiments, the injectable hydrogel can be formed in situ by Michael-type addition crosslinking. In other embodiments, the injectable hydrogel can be formed in situ by a shear-thinning gelation mechanism. In other embodiments, the injectable hydrogel can be formed in situ by a thermosensitive gelation mechanism.

Injectable, biodegradable hydrogels can be formed in situ by copper-free click chemistry comprising placing a first predominantly hydrophilic polymer comprising at least two functional azide group moieties and a second predominantly hydrophilic polymer containing at least two functional alkyne group moieties within a subject in a manner that permits the functional groups of the first polymer and the functional groups of the second polymer to react via a copper-free azide-alkyne cyclo-addition mechanism to form an in situ crosslinked hydrogel, wherein the resulting hydrogel undergoes hydrolysis or enzymatic cleavage under physiologically relevant conditions.

Injectable, biodegradable hydrogels can be formed in situ by a Michael-type addition reaction comprising placing a first predominantly hydrophilic polymer comprising at least two functional alkene group moieties and a second predominantly hydrophilic polymer containing at least two functional reduced thiol group moieties within a subject in a manner that permits the functional groups of the first polymer and the functional groups of the second polymer to react via a Michael-type addition reaction mechanism to form an in situ crosslinked hydrogel, wherein the resulting hydrogel undergoes hydrolysis or enzymatic cleavage under physiologically relevant conditions. Reduced thiol groups are necessary and are produced by reaction with a reducing agent (e.g. reduced glutathione) prior to or during the in situ reaction.

When the components for forming the present hydrogels are, for example, introduced into a human or animal subject, the resulting hydrogels can provide structural support, delivery of an active agent, or both, over a desired period of time. By selection of the materials and conditions under which the present hydrogels are formed, it is possible to form a hydrogel having specific degradation characteristics in situ that are optimal for the desired function of the hydrogel. When the hydrogel contains an active agent, the rate and profile of degradation of the hydrogel will influence the profile of the delivery of the active agent to the site to which the hydrogel is delivered. When the hydrogel is intended to provide structural support to the delivery site, the degradation profile will determine the time over which the structural support is present. Thus, these biocompatible, biodegradable injectable hydrogels that are designed to both self-assemble in situ and have tunable degradation characteristics have the ability to deliver an active agent, provide structural support, or both over a desired period of time. These characteristics permit treatment in a manner and over time period that is optimized for the treatment of spinal cord injury.

Suitable agents capable of specifically reducing TNF-α signaling include a TNF-α inhibitor, a protein that specifically binds to TNF-α, an anti-inflammatory cytokine, or any combination thereof. In some embodiments, the one or more agents capable of specifically reducing TNF-α signaling comprise a TNF-α inhibitor. In some embodiments, the one or more agents capable of specifically reducing TNF-α signaling comprise a protein that specifically binds TNF-α. In some aspects, the protein that specifically binds TNF-α is an antibody. In some embodiments, the one or more agents capable of specifically reducing TNF-α signaling comprise an anti-inflammatory cytokine.

Suitable TNF-α inhibitors include, but are not limited to, Etanercept (Enbrel®), Infliximab (REMICADE®), Adalimumab (HUMIRA®), Certolizumab pegol (CIMZIA®), Pentoxifylline (TRENTAL®), methotrexate, pirfenidone, Bupropion (WELLBUTRIN®), or any combination thereof.

Suitable proteins that specifically bind TNF-α include, but are not limited to, Etanercept (Enbrel®), Infliximab (REMICADE®), Adalimumab (HUMIRA®), Certolizumab pegol (CIMZIA®), or any combination thereof.

Suitable agents for use in the disclosed compositions include agents that reduce TNF-α signaling independent of modulating the cell cycle.

The one or more agents can be exposed on the surface of the biodegradable carrier, incorporated within the biodegradable carrier, or both. In some embodiments, the one or more of said agents are exposed on the surface of the biodegradable carrier. The exposed agent can bind to and inactivate TNF-α through the sequestration of, and/or degradation of, soluble TNF-α. For example, the exposed agent can bind TNF-α and the biodegradable carrier can subsequently be internalized by a cell, via endocytosis or other means known in the art, whereby the TNF-α can be delivered to the lysosomes for degradation. In some embodiments, the agent exposed on the surface of the biodegradable carrier is a protein that specifically binds TNF-α, such as an antibody.

In some embodiments, the one or more agents are incorporated within the biodegradable carrier.

In other embodiments, the one or more of said agents are exposed on the surface of the biodegradable carrier and incorporated within the biodegradable carrier. In some aspects, the one or more agents incorporated within the biodegradable carrier is an anti-inflammatory cytokine and the one or more agents exposed on the surface of the biodegradable carrier comprise a protein that specifically binds TNF-α. In some aspects, the one or more agents exposed on the surface of the biodegradable carrier and the one or more agents incorporated within the biodegradable carrier is a protein that specifically binds TNF-α, a TNF-α inhibitor, or any combination thereof. In some aspects, the one or more agents exposed on the surface of the biodegradable carrier is a protein that specifically binds TNF-α and the one or more agents incorporated within the biodegradable carrier is a protein that specifically binds TNF-α. In some aspects, the one or more agents exposed on the surface of the biodegradable carrier is a TNF-α inhibitor and the one or more agents incorporated within the biodegradable carrier is a TNF-α inhibitor. In some aspects, the one or more agents exposed on the surface of the biodegradable carrier is a protein that specifically binds TNF-α and the one or more agents incorporated within the biodegradable carrier is a TNF-α inhibitor. In some aspects, the one or more agents exposed on the surface of the biodegradable carrier is a TNF-α inhibitor and the one or more agents incorporated within the biodegradable carrier is a protein that specifically binds TNF-α.

In some embodiments, the composition can further comprise one or more anti-inflammatory cytokines. Numerous anti-inflammatory cytokines are known to those skilled in the art, including, but not limited to, IL-10, IL-4, or TGF-β. In some aspects the one or more anti-inflammatory cytokines is IL-10. In other aspects the one or more anti-inflammatory cytokines is IL-4.

The one or more anti-inflammatory cytokines can be exposed on the surface of the biodegradable carrier, incorporated within the biodegradable carrier, or both. In some embodiments, the one or more anti-inflammatory cytokines are incorporated within the biodegradable carrier.

In some aspects, the biodegradable carrier can provide 3-D architecture for tissue engineering purposes while the one or more agents exposed on the surface of or incorporated within the biodegradable carrier can enable the clearance of TNF-α.

The biodegradable carrier can be designed to begin to degrade within any suitable time frame following administration of a composition to a subject. In some embodiments, the biodegradable carrier can begin to degrade from the time of being administered to about 21 days following being administered of the composition to a subject.

The biodegradable carrier can begin to degrade within about 21 days of being administered to a subject. The biodegradable carrier can begin to degrade within about 14 days of being administered to a subject. The biodegradable carrier can begin to degrade within about 10 days of being administered to a subject. The biodegradable carrier can begin to degrade within about 7 days of being administered to a subject. The biodegradable carrier can begin to degrade within about 5 days of being administered to a subject. The biodegradable carrier can begin to degrade within about 3 days of being administered to a subject. The biodegradable carrier can begin to degrade within about 1 day of being administered to a subject. The biodegradable carrier can begin to degrade at the time of being administered to a subject.

Alternatively, the biodegradable carrier can begin to degrade within a short period of time. In some instances the biodegradable carrier can begin to degrade within as few as 48 hours of being administered to a subject. In some instances the biodegradable carrier can begin to degrade within as few as 36 hours of being administered to a subject. In some instances the biodegradable carrier can begin to degrade within as few as 24 hours of being administered to a subject. In some instances the biodegradable carrier can begin to degrade within as few as 12 hours of being administered to a subject. In some instances the biodegradable carrier can begin to degrade within as few as 6 hours of being administered to a subject. In some instances the biodegradable carrier can begin to degrade instantaneously upon being administered to a subject.

Degradation of the biodegradable carrier can lead to the release of, and/or delivery of, the one or more agents, thus providing a therapeutically effective dose of the one or more agents to the subject. In some embodiments, the biodegradable carrier provides a therapeutically effective dose of the agents for up to about 21 days. In some embodiments, the biodegradable carrier provides a therapeutically effective dose of the agents for up to about 18 days. In some embodiments, the biodegradable carrier provides a therapeutically effective dose of the agents for up to about 14 days. In some embodiments, the biodegradable carrier provides a therapeutically effective dose of the agents for up to about 12 days. In some embodiments, the biodegradable carrier provides a therapeutically effective dose of the agents for up to about 10 days. In some embodiments, the biodegradable carrier provides a therapeutically effective dose of the agents for up to about 9 days. In some embodiments, the biodegradable carrier provides a therapeutically effective dose of the agents for up to about 8 days. In some embodiments, the biodegradable carrier provides a therapeutically effective dose of the agents for up to about 7 days. In some embodiments, the biodegradable carrier provides a therapeutically effective dose of the agents for up to about 6 days. In some embodiments, the biodegradable carrier provides a therapeutically effective dose of the agents for up to about 5 days. In some embodiments, the biodegradable carrier provides a therapeutically effective dose of the agents for up to about 4 days. In some embodiments, the biodegradable carrier provides a therapeutically effective dose of the agents for up to about 3 days. In some embodiments, the biodegradable carrier provides a therapeutically effective dose of the agents for up to about 2 days. In some embodiments, the biodegradable carrier provides a therapeutically effective dose of the agents for up to about 1 day.

The biodegradable carrier can deliver a therapeutically effective dose of the one or more agents from about day 1 to about day 21 of being administered to a subject. The biodegradable carrier can deliver a therapeutically effective dose of the one or more agents from about day 1 to about day 14 of being administered to a subject. The biodegradable carrier can deliver a therapeutically effective dose of the one or more agents from about day 1 to about day 7 of being administered to a subject. The biodegradable carrier can deliver a therapeutically effective dose of the one or more agents from about day 1 to about day 3 of being administered to a subject. The biodegradable carrier can deliver a therapeutically effective dose of the one or more agents from about day 3 to about day 21 of being administered to a subject. The biodegradable carrier can deliver a therapeutically effective dose of the one or more agents from about day 3 to about day 14 of being administered to a subject. The biodegradable carrier can deliver a therapeutically effective dose of the one or more agents from about day 3 to about day 7 of being administered to a subject. The biodegradable carrier can deliver a therapeutically effective dose of the one or more agents from about day 7 to about day 21 of being administered to a subject. The biodegradable carrier can deliver a therapeutically effective dose of the one or more agents from about day 7 to about day 14 of being administered to a subject. The biodegradable carrier can deliver a therapeutically effective dose of the one or more agents from about day 7 to about day 10 of being administered to a subject. The biodegradable carrier can deliver a therapeutically effective dose of the one or more agents from about day 14 to about day 21 of being administered to a subject.

Alternatively, the biodegradable carrier can deliver a therapeutically effective dose of the one or more agents within a short period of time. In some instances the biodegradable carrier can deliver a therapeutically effective dose of the one or more agents within as few as 48 hours of being administered to a subject. In some instances the biodegradable carrier can deliver a therapeutically effective dose of the one or more agents within as few as 36 hours of being administered to a subject. In some instances the biodegradable carrier can deliver a therapeutically effective dose of the one or more agents within as few as 24 hours of being administered to a subject. In some instances the biodegradable carrier can deliver a therapeutically effective dose of the one or more agents within as few as 12 hours of being administered to a subject. In some instances the biodegradable carrier can deliver a therapeutically effective dose of the one or more agents within as few as 6 hours of being administered to a subject. In some instances the biodegradable carrier can deliver a therapeutically effective dose of the one or more agents within as few as 3 hours of being administered to a subject. In some instances the biodegradable carrier can deliver a therapeutically effective dose of the one or more agents within 1 hour of being administered to a subject. In some instances the biodegradable carrier can deliver a therapeutically effective dose of the one or more agents instantaneously upon being administered to a subject.

The therapeutically effective dose of the one or more agents can be delivered to the site of injury, can be released systemically, or can be delivered to the site of injury and released systemically. For example, in some embodiments, the one or more agents can be delivered to the spinal cord.

Pharmaceutical agents may also be included in the compositions described herein. In some aspects, the pharmaceutical agents may stabilize the composition, allow it to be readily administered to a subject, increase its ability to specifically reduce TNF-α signaling, or otherwise make the composition suitable for therapeutic use in a subject. Accordingly, the described composition may further comprise a pharmaceutically acceptable carrier or excipient, as would be known to an individual skilled in the relevant art. In view of the inclusion of pharmaceutical agents in some of the described compositions, disclosed herein are also pharmaceutical compositions having one or more agents capable of specifically reducing TNF-α signaling and a biodegradable carrier, as provided herein. The described pharmaceutical compositions for delivery or injection of the described compositions may be administered to a subject in order to maintain the ability to specifically reduce TNF-α signaling in the subject over a prolonged period of time. For example, composition viscosity and concentration of the one or more agents capable of specifically reducing TNF-α signaling may be altered to increase the half-life of composition's active ingredients.

The described pharmaceutical compositions may be formulated as any of various preparations that are known and suitable in the art, including those described and exemplified herein. In some embodiments, the pharmaceutical compositions are aqueous formulations. Aqueous solutions may be prepared by admixing the described compositions in water or suitable physiologic buffer, and optionally adding suitable colorants, preservatives, stabilizing and thickening agents, ions such as calcium or magnesium, and the like as desired. Aqueous suspensions may also be made by dispersing the described compositions in water or physiologic buffer with viscous material, such as natural or synthetic gums, resins, methylcellulose, sodium carboxymethylcellulose, and other well-known suspending agents. Also included are liquid formulations and solid form preparations which are intended to be converted, shortly before use, to liquid preparations. Such liquids include solutions, suspensions, syrups, slurries, and emulsions. Liquid preparations may be prepared by conventional means with pharmaceutically acceptable additives such as suspending agents (e.g., sorbitol syrup, cellulose derivatives or hydrogenated edible fats or oils); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). These preparations may contain, in addition to the active agent, stabilizers, buffers, dispersants, thickeners, solubilizing agents, and the like. The compositions may be in powder or lyophilized form for constitution with a suitable vehicle such as sterile water, physiological buffer, or saline solution before use. The compositions may be formulated for injection into a subject. For injection, the compositions described may be formulated in aqueous solutions such as water, or in physiologically compatible buffers such as Hanks's solution, Ringer's solution, physiological saline buffer, or artificial cerebral spinal fluid. The solution may contain one or more formulatory agents such as suspending, stabilizing or dispersing agents. Injection formulations may also be prepared as solid form preparations which are intended to be converted, shortly before use, to liquid form preparations suitable for injection, for example, by constitution with a suitable vehicle, such as sterile water, saline solution, or artificial cerebral spinal fluid before use.

Also provided herein are methods of treating inflammation in a subject having a spinal cord injury comprising administering to said subject a composition comprising one or more agents capable of specifically reducing TNF-α signaling and a biodegradable carrier.

In some embodiments, the one or more agents are capable of specifically reducing TNF-α signaling by directly reducing TNF-α signaling. For example, in some aspects, the one or more agents can inhibit TNF-α directly. In other aspects, the one or more agents can inhibit proteins and/or factors upstream of TNF-α. In other aspects, the one or more agents can inhibit proteins and/or factors downstream of TNF-α.

The disclosed compositions can be administered to a subject by a number of routes, including, but not limited to, intrathecally, intravenously, intra-arterially, transdermally, subcutaneously, topically, or any combination thereof. In some embodiments, the composition can be administered to the spinal cord of the subject. For example, the composition can be administered by direct injection into the spinal cord of the subject. In some aspects, the composition can be administered by surgically implanting the composition into the spinal cord of the subject.

As the injuries suitable for treatment include traumatic bodily injuries that affect the spinal cord, the described methods may be carried out when the temperature of the body or spinal region has been lowered. In some embodiments the described compositions may be administered when the spinal cord of the subject is from about 96° F. to about 85° F. In some embodiments the described compositions may be administered when the spinal cord of the subject is about 96° F., about 95° F., about 94° F., about 93° F., about 92° F., about 91° F., about 90° F., about 89° F., about 88° F., or about 87° F. Also, because rapid treatment is often desirable for spinal cord injuries, the described methods may be carried out within about 2 hours of a subject's spinal cord injury. In some embodiments the described methods may be carried out within about 4 hours of a subject's spinal cord injury. In some embodiments the described methods may be carried out within about 6 hours of a subject's spinal cord injury. In some embodiments the described methods may be carried out within about 12 hours of a subject's spinal cord injury. In some embodiments the described methods may be carried out within about 18 hours of a subject's spinal cord injury. In some embodiments the described methods may be carried out within about 24 hours of a subject's spinal cord injury. In some embodiments the described methods may be carried out within about 36 hours of a subject's spinal cord injury. In some embodiments the described methods may be carried out within about 48 hours of a subject's spinal cord injury. In some embodiments the described methods may be carried out within about 72 hours of a subject's spinal cord injury. In some embodiments, the described methods can be carried out from the time of a subject's spinal cord injury to about 1 week after a subject's spinal cord injury. In other embodiments, the described methods can be carried out from the time of a subject's spinal cord injury to about 72 hours after a subject's spinal cord injury. In other embodiments, the described methods can be carried out from the time of a subject's spinal cord injury to about 48 hours after a subject's spinal cord injury. In other embodiments, the described methods can be carried out from the time of a subject's spinal cord injury to about 24 hours after a subject's spinal cord injury. In some embodiments, the described methods can be carried out from about 24 hours after a subject's spinal cord injury to about 1 week after a subject's spinal cord injury. In other embodiments, the described methods can be carried out from about 24 hours after a subject's spinal cord injury to about 72 hours after a subject's spinal cord injury. In other embodiments, the described methods can be carried out from about 24 hours after a subject's spinal cord injury to about 48 hours after a subject's spinal cord injury. In some embodiments, the described methods can be carried out from about 48 hours after a subject's spinal cord injury to about 1 week after a subject's spinal cord injury. In other embodiments, the described methods can be carried out from about 48 hours after a subject's spinal cord injury to about 72 hours after a subject's spinal cord injury.

In some embodiments the described methods may be carried out within about 72 hours of initiation of treatment for a subject's spinal cord injury. In some embodiments the described methods may be carried out within about 48 hours of initiation of treatment for a subject's spinal cord injury. In some embodiments the described methods may be carried out within about 24 hours of initiation of treatment for a subject's spinal cord injury. In some embodiments the described methods may be carried out within about 18 hours of initiation of treatment for a subject's spinal cord injury. In some embodiments the described methods may be carried out within about 12 hours of initiation of treatment for a subject's spinal cord injury. In some embodiments the described methods may be carried out within about 6 hours of initiation of treatment for a subject's spinal cord injury. In some embodiments the described methods may be carried out within about 4 hours of initiation of treatment for a subject's spinal cord injury. In some embodiments the described methods may be carried out within about 3 hours of initiation of treatment for a subject's spinal cord injury. In some embodiments the described methods may be carried out within about 2 hours of initiation of treatment for a subject's spinal cord injury. In some embodiments the described methods may be carried out within about 1 hour of initiation of treatment for a subject's spinal cord injury. In some embodiments the described methods may be carried out less than 1 hour after initiation of treatment for a subject's spinal cord injury.

Also provided herein are kits for producing a composition comprising one or more agents capable of specifically reducing TNF-α signaling and a biodegradable carrier, the kits comprising: one or more agents capable of specifically reducing TNF-α signaling; a biodegradable carrier; and instructions for producing said composition.

### Compositions Comprising One or More Agents Capable of Modulating MCP-1 Signaling

Disclosed herein are compositions for inhibiting inflammation in a subject with a spinal cord injury comprising, one or more agents capable of modulating MCP-1 signaling and a biodegradable carrier.

Suitable biodegradable carriers include, but are not limited to, a microparticle, a nanoparticle, a hydrogel, or any combination thereof.

Biodegradable carriers can comprise synthetically derived polymers, including, biodegradable polymers. Exemplary polymers include, but are not limited to, poly(lactides) (PLA), poly(glycolides) (PGA), poly(lactide-co-glycolides) (PLGA), poly(ethylene glycols)(PEG), or any combination thereof. In some embodiments, the synthetically derived biodegradable polymer can be poly(lactic-co-glycolic acid) (PLGA), having a lactic acid and glycolic acid content ranging from 0-100% for each monomer. For example, in some aspects, the biodegradable polymer can be a 50:50 PLGA, where 50:50 refers to the ratio of lactic to glycolic acid. In some embodiments, the biodegradable carrier comprises or consists of a copolymer. For example, in some embodiments, the biodegradable polymer can be a copolymer of poly(ethylene glycol) (PEG) and poly(lactic-co-glycolic acid) (PLGA), having a lactic acid and glycolic acid content ranging from 0-100% for each monomer. Further, in some embodiments, the biodegradable carrier can be a microparticle and/or nanoparticle comprising 50:50 PLGA. In other embodiments, the biodegradable carrier can be a microparticle and/or nanoparticle comprising a copolymer of 50:50 PLGA and PEG. In yet other embodiments, the biodegradable carrier can be a hydrogel comprising PEGs and/or copolymers of PEG and PLGA.

Exemplary biodegradable microparticles and/or nanoparticles can be fabricated using processing techniques known by those skilled in the art, including, but not limited to, emulsification, precipitation, or spray drying. In some embodiments, the microparticles and/or nanoparticles can be fabricated by emulsification. In other embodiment, the microparticles and/or nanoparticles can be fabricated by precipitation or nanoprecipitation, respectively. In yet other embodiments, the microparticles and/or nanoparticles can be fabricated by spray drying.

Injectable, biodegradable hydrogels can be formed in situ by copper-free click chemistry comprising placing a first predominantly hydrophilic polymer comprising at least two functional azide group moieties and a second predominantly hydrophilic polymer containing at least two functional alkyne group moieties within a subject in a manner that permits the functional groups of the first polymer and the functional groups of the second polymer to react via a copper-free azide-alkyne cyclo-addition mechanism to form an in situ crosslinked hydrogel, wherein the resulting hydrogel undergoes hydrolysis or enzymatic cleavage under physiologically relevant conditions.

Injectable, biodegradable hydrogels can be formed in situ by a Michael-type addition reaction comprising placing a first predominantly hydrophilic polymer comprising at least two functional alkene group moieties and a second predominantly hydrophilic polymer containing at least two functional reduced thiol group moieties within a subject in a manner that permits the functional groups of the first polymer and the functional groups of the second polymer to react via a Michael-type addition reaction mechanism to form an in situ crosslinked hydrogel, wherein the resulting hydrogel undergoes hydrolysis or enzymatic cleavage under physiologically relevant conditions. Reduced thiol groups are necessary and are produced by reaction with a reducing agent (e.g. reduced glutathione) prior to or during the in situ reaction.

When the components for forming the present hydrogels are, for example, introduced into a human or animal subject, the resulting hydrogels can provide structural support, delivery of an active agent, or both, over a desired period of time. By selection of the materials and conditions under which the present hydrogels are formed, it is possible to form a hydrogel having specific degradation characteristics in situ that are optimal for the desired function of the hydrogel. When the hydrogel contains an active agent, the rate and profile of degradation of the hydrogel will influence the profile of the delivery of the active agent to the site to which the hydrogel is delivered. When the hydrogel is intended to provide structural support to the delivery site, the degradation profile will determine the time over which the structural support is present. Thus, these biocompatible, biodegradable injectable hydrogels that are designed to both self-assemble in situ and have tunable degradation characteristics have the ability to deliver an active agent, provide structural support, or both over a desired period of time. These characteristics permit treatment in a manner and over time period that is optimized for the treatment of spinal cord injury.

Suitable agents capable of modulating MCP-1 signaling include, but are not limited to, a JNK inhibitor, a TNF-α inhibitor, a protein that specifically binds TNF-α, a protein that specifically binds MCP-1, a non-selective COX inhibitor, a selective COX inhibitor, a COX-2 inhibitor, a nonsteroidal anti-inflammatory drug (NSAID), a tetracycline, an anti-inflammatory cytokine, methotrexate, pirfenidone, or any combination thereof.

JNK inhibitors include, but are not limited to, one or more of the following, SP600125, Bentamapimod, RWJ67657, TCSJNK60, SU3327, CC-401, or BI78D3. In some embodiments, the JNK inhibitor is SP600125.

Proteins that specifically binds TNF-α include, but are not limited to, one or more of Etanercept (Enbrel®), Infliximab (REMICADE®), Adalimumab (HUMIRA®), Certolizumab pegol (CIMZIA®), or any combination thereof.

TNF-α inhibitors include, but are not limited to, Pentoxifylline (TRENTAL®), methotrexate, pirfenidone, Bupropion (WELLBUTRIN®), or any combination thereof.

Proteins that specifically binds MCP-1 include an antibody. In some embodiments, the protein that specifically binds MCP-1 is ABN912.

COX inhibitors include, but are not limited to, one or more of the following, celecoxib (Celebrex®), Vioxx®, Bextra®, Prexige®, Arcoxia®, curcumin, Deguelin, nifllumic acid, ibuprofen (Advil®), or naproxen (Aleve®). In some embodiments, the COX inhibitor is a COX-2 inhibitor. In some embodiments, the COX-2 inhibitor celecoxib (Celebrex®). In other embodiments, the COX-2 inhibitor is curcumin. In some embodiments, the COX-2 inhibitor is Vioxx.

The COX inhibitor can be a NSAID. For example, in some aspects, the NSAID can be ibuprofen. In other aspects, the NSAID can be naproxen. In yet other aspects, the NSAID can be a combination of ibuprofen and naproxen.

Suitable tetracylines include minocycline, doxycycline, or any combination thereof.

Suitable agents for use in the disclosed compositions include agents that modulate MCP-1 signaling independent of modulating the cell cycle.

The one or more agents capable of modulating MCP-1 signaling can be exposed on the surface of the biodegradable carrier, incorporated within the biodegradable carrier, or both. In some embodiments, the one or more of said agents are exposed on the surface of the biodegradable carrier. For example, in some aspects, the one or more agents exposed on the surface of the biodegradable carrier can be a TNF-α binding proteins, such as an antibody. In other aspects, the one or more agents exposed on the surface of the biodegradable carrier can be an MCP-1 binding protein. In other aspects, the one or more agents exposed on the surface of the biodegradable carrier can be a TNF-α binding protein and an MCP-1 binding protein. The exposed TNF-α binding proteins can bind to and inactivate TNF-α through the sequestration of, and/or degradation of, circulating TNF-α by, for example, TNF-α binding and the subsequent internalization and trafficking of the biodegradable carrier to the lysosomes. In some aspects, the one or more agents capable of modulating MCP-1 signaling comprise a TNF-α inhibitor.

In other embodiments, the one or more agents can be incorporated within the biodegradable carrier.

In yet other embodiments, the one or more agents can be exposed on the surface of the biodegradable carrier and incorporated within the biodegradable carrier. For example, in some aspects, the one or more agents incorporated within the biodegradable carrier can be an anti-inflammatory cytokine and the one or more agents exposed on the surface of the biodegradable carrier can be a protein that specifically binds TNF-α. For example, in some aspects IL-10 can be incorporated within the biodegradable carrier and a protein that specifically binds TNF-α, such as an antibody, can be exposed on the surface of the biodegradable carrier. In other embodiments, the one or more agents incorporated within the biodegradable carrier can be an anti-inflammatory cytokine and the one or more agents exposed on the surface of the biodegradable carrier can be a protein that specifically binds MCP-1. In yet other embodiments, the one or more agents incorporated within the biodegradable carrier can be a TNF-α inhibitor, a COX inhibitor, a COX-2 inhibitor, or a tetracycline, and the one or more agents exposed on the surface of the biodegradable carrier can be a protein that specifically binds TNF-α. In yet other embodiments, the one or more agents incorporated within the biodegradable carrier can be a TNF-α inhibitor, a COX inhibitor, a COX-2 inhibitor, or a tetracycline, and the one or more agents exposed on the surface of the biodegradable carrier can be a protein that specifically binds MCP-1.

In some embodiments, the composition can further comprise one or more anti-inflammatory cytokines. Numerous anti-inflammatory cytokines are known to those skilled in the art, including, but not limited to, IL-10, IL-4, or TGF-β. In some aspects the one or more anti-inflammatory cytokines is IL-10. In other aspects the one or more anti-inflammatory cytokines is IL-4.

In some aspects, the biodegradable carrier can provide 3-D architecture for tissue engineering purposes while the one or more agents exposed on the surface of, or incorporated within the biodegradable carrier can enable the modulation of MCP-1 signaling.

The biodegradable carrier can be designed to begin to degrade within any suitable time frame following administration of a composition to a subject. In some embodiments, the biodegradable carrier can begin to degrade from the time of being administered to about 21 days following being administered of the composition to a subject.

The biodegradable carrier can begin to degrade within about 21 days of being administered to a subject. The biodegradable carrier can begin to degrade within about 14 days of being administered to a subject. The biodegradable carrier can begin to degrade within about 10 days of being administered to a subject. The biodegradable carrier can begin to degrade within about 7 days of being administered to a subject. The biodegradable carrier can begin to degrade within about 5 days of being administered to a subject. The biodegradable carrier can begin to degrade within about 3 days of being administered to a subject. The biodegradable carrier can begin to degrade within about 1 day of being administered to a subject. The biodegradable carrier can begin to degrade at the time of being administered to a subject.

Alternatively, the biodegradable carrier can begin to degrade within a short period of time. In some instances the biodegradable carrier can begin to degrade within as few as 48 hours of being administered to a subject. In some instances the biodegradable carrier can begin to degrade within as few as 36 hours of being administered to a subject. In some instances the biodegradable carrier can begin to degrade within as few as 24 hours of being administered to a subject. In some instances the biodegradable carrier can begin to degrade within as few as 12 hours of being administered to a subject. In some instances the biodegradable carrier can begin to degrade within as few as 6 hours of being administered to a subject. In some instances the biodegradable carrier can begin to degrade instantaneously upon being administered to a subject.

Degradation of the biodegradable carrier can lead to the release of, and/or delivery of, the one or more agents, thus providing a therapeutically effective dose of the one or more agents to the subject. In some embodiments, the biodegradable carrier provides a therapeutically effective dose of the agents for up to about 21 days. In some embodiments, the biodegradable carrier provides a therapeutically effective dose of the agents for up to about 18 days. In some embodiments, the biodegradable carrier provides a therapeutically effective dose of the agents for up to about 14 days. In some embodiments, the biodegradable carrier provides a therapeutically effective dose of the agents for up to about 12 days. In some embodiments, the biodegradable carrier provides a therapeutically effective dose of the agents for up to about 10 days. In some embodiments, the biodegradable carrier provides a therapeutically effective dose of the agents for up to about 9 days. In some embodiments, the biodegradable carrier provides a therapeutically effective dose of the agents for up to about 8 days. In some embodiments, the biodegradable carrier provides a therapeutically effective dose of the agents for up to about 7 days. In some embodiments, the biodegradable carrier provides a therapeutically effective dose of the agents for up to about 6 days. In some embodiments, the biodegradable carrier provides a therapeutically effective dose of the agents for up to about 5 days. In some embodiments, the biodegradable carrier provides a therapeutically effective dose of the agents for up to about 4 days. In some embodiments, the biodegradable carrier provides a therapeutically effective dose of the agents for up to about 3 days. In some embodiments, the biodegradable carrier provides a therapeutically effective dose of the agents for up to about 2 days. In some embodiments, the biodegradable carrier provides a therapeutically effective dose of the agents for up to about 1 day.

The biodegradable carrier can deliver a therapeutically effective dose of the one or more agents from about day 1 to about day 21 of being administered to a subject. The biodegradable carrier can deliver a therapeutically effective dose of the one or more agents from about day 1 to about day 14 of being administered to a subject. The biodegradable carrier can deliver a therapeutically effective dose of the one or more agents from about day 1 to about day 7 of being administered to a subject. The biodegradable carrier can deliver a therapeutically effective dose of the one or more agents from about day 1 to about day 3 of being administered to a subject. The biodegradable carrier can deliver a therapeutically effective dose of the one or more agents from about day 3 to about day 21 of being administered to a subject. The biodegradable carrier can deliver a therapeutically effective dose of the one or more agents from about day 3 to about day 14 of being administered to a subject. The biodegradable carrier can deliver a therapeutically effective dose of the one or more agents from about day 3 to about day 7 of being administered to a subject. The biodegradable carrier can deliver a therapeutically effective dose of the one or more agents from about day 7 to about day 21 of being administered to a subject. The biodegradable carrier can deliver a therapeutically effective dose of the one or more agents from about day 7 to about day 14 of being administered to a subject. The biodegradable carrier can deliver a therapeutically effective dose of the one or more agents from about day 7 to about day 10 of being administered to a subject. The biodegradable carrier can deliver a therapeutically effective dose of the one or more agents from about day 14 to about day 21 of being administered to a subject.

Alternatively, the biodegradable carrier can deliver a therapeutically effective dose of the one or more agents within a short period of time. In some instances the biodegradable carrier can deliver a therapeutically effective dose of the one or more agents within as few as 48 hours of being administered to a subject. In some instances the biodegradable carrier can deliver a therapeutically effective dose of the one or more agents within as few as 36 hours of being administered to a subject. In some instances the biodegradable carrier can deliver a therapeutically effective dose of the one or more agents within as few as 24 hours of being administered to a subject. In some instances the biodegradable carrier can deliver a therapeutically effective dose of the one or more agents within as few as 12 hours of being administered to a subject. In some instances the biodegradable carrier can deliver a therapeutically effective dose of the one or more agents within as few as 6 hours of being administered to a subject. In some instances the biodegradable carrier can deliver a therapeutically effective dose of the one or more agents within as few as 3 hours of being administered to a subject. In some instances the biodegradable carrier can deliver a therapeutically effective dose of the one or more agents within 1 hour of being administered to a subject. In some instances the biodegradable carrier can deliver a therapeutically effective dose of the one or more agents instantaneously upon being administered to a subject.

The therapeutically effective dose of the one or more agents can be delivered to the site of injury, can be released systemically, or can be delivered to the site of injury and released systemically. For example, in some embodiments, the one or more agents can be delivered to the spinal cord.

Pharmaceutical agents may also be included in the compositions described herein. In some aspects, the pharmaceutical agents may stabilize the composition, allow it to be readily administered to a subject, increase its ability to modulate MCP-1 signaling, or otherwise make the composition suitable for therapeutic use in a subject. Accordingly, the described composition may further comprise a pharmaceutically acceptable carrier or excipient, as would be known to an individual skilled in the relevant art. In view of the inclusion of pharmaceutical agents in some of the described compositions, disclosed herein are also pharmaceutical compositions having one or more agents capable of modulating MCP-1 signaling and a biodegradable carrier, as provided herein. The described pharmaceutical compositions for delivery or injection of the described compositions may be administered to a subject in order to maintain the ability to modulate MCP-1 signaling in the subject over a prolonged period of time. For example, composition viscosity and concentration of the one or more agents capable of modulating MCP-1 signaling may be altered to increase the half-life of composition's active ingredients.

The described pharmaceutical compositions may be formulated as any of various preparations that are known and suitable in the art, including those described and exemplified herein. In some embodiments, the pharmaceutical compositions are aqueous formulations. Aqueous solutions may be prepared by admixing the described compositions in water or suitable physiologic buffer, and optionally adding suitable colorants, preservatives, stabilizing and thickening agents, ions such as calcium or magnesium, and the like as desired. Aqueous suspensions may also be made by dispersing the described compositions in water or physiologic buffer with viscous material, such as natural or synthetic gums, resins, methylcellulose, sodium carboxymethylcellulose, and other well-known suspending agents. Also included are liquid formulations and solid form preparations which are intended to be converted, shortly before use, to liquid preparations. Such liquids include solutions, suspensions, syrups, slurries, and emulsions. Liquid preparations may be prepared by conventional means with pharmaceutically acceptable additives such as suspending agents (e.g., sorbitol syrup, cellulose derivatives or hydrogenated edible fats or oils); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). These preparations may contain, in addition to the active agent, stabilizers, buffers, dispersants, thickeners, solubilizing agents, and the like. The compositions may be in powder or lyophilized form for constitution with a suitable vehicle such as sterile water, physiological buffer, or saline solution before use. The compositions may be formulated for injection into a subject. For injection, the compositions described may be formulated in aqueous solutions such as water, or in physiologically compatible buffers such as Hanks's solution, Ringer's solution, physiological saline buffer, or artificial cerebral spinal fluid. The solution may contain one or more formulatory agents such as suspending, stabilizing or dispersing agents. Injection formulations may also be prepared as solid form preparations which are intended to be converted, shortly before use, to liquid form preparations suitable for injection, for example, by constitution with a suitable vehicle, such as sterile water, saline solution, or artificial cerebral spinal fluid before use.

Also disclosed herein are methods of treating inflammation in a subject having spinal cord injury comprising administering to said subject a composition comprising one or more agents capable of modulating MCP-1 signaling and a biodegradable carrier.

The disclosed compositions can be administered to a subject by a number of routes, including, but not limited to, intrathecally, intravenously, intra-arterially, transdermally, subcutaneously, topically, or any combination thereof. In some embodiments, the composition can be administered to the spinal cord of the subject. For example, the composition can be administered by direct injection into the spinal cord of the subject. In some aspects, the composition can be administered by surgically implanting the composition into the spinal cord of the subject.

As the injuries suitable for treatment include traumatic bodily injuries that affect the spinal cord, the described methods may be carried out when the temperature of the body or spinal region has been lowered. In some embodiments the described compositions may be administered when the spinal cord of the subject is from about 96° F. to about 85° F. In some embodiments the described compositions may be administered when the spinal cord of the subject is about 96° F., about 95° F., about 94° F., about 93° F., about 92° F., about 91° F., about 90° F., about 89° F., about 88° F., or about 87° F. Also, because rapid treatment is often desirable for spinal cord injuries, the described methods may be carried out within about 2 hours of a subject's spinal cord injury. In some embodiments the described methods may be carried out within about 4 hours of a subject's spinal cord injury. In some embodiments the described methods may be carried out within about 6 hours of a subject's spinal cord injury. In some embodiments the described methods may be carried out within about 12 hours of a subject's spinal cord injury. In some embodiments the described methods may be carried out within about 18 hours of a subject's spinal cord injury. In some embodiments the described methods may be carried out within about 24 hours of a subject's spinal cord injury. In some embodiments the described methods may be carried out within about 36 hours of a subject's spinal cord injury. In some embodiments the described methods may be carried out within about 48 hours of a subject's spinal cord injury. In some embodiments the described methods may be carried out within about 72 hours of a subject's spinal cord injury. In some embodiments, the described methods can be carried out from the time of a subject's spinal cord injury to about 1 week after a subject's spinal cord injury. In other embodiments, the described methods can be carried out from the time of a subject's spinal cord injury to about 72 hours after a subject's spinal cord injury. In other embodiments, the described methods can be carried out from the time of a subject's spinal cord injury to about 48 hours after a subject's spinal cord injury. In other embodiments, the described methods can be carried out from the time of a subject's spinal cord injury to about 24 hours after a subject's spinal cord injury. In some embodiments, the described methods can be carried out from about 24 hours after a subject's spinal cord injury to about 1 week after a subject's spinal cord injury. In other embodiments, the described methods can be carried out from about 24 hours after a subject's spinal cord injury to about 72 hours after a subject's spinal cord injury. In other embodiments, the described methods can be carried out from about 24 hours after a subject's spinal cord injury to about 48 hours after a subject's spinal cord injury. In some embodiments, the described methods can be carried out from about 48 hours after a subject's spinal cord injury to about 1 week after a subject's spinal cord injury. In other embodiments, the described methods can be carried out from about 48 hours after a subject's spinal cord injury to about 72 hours after a subject's spinal cord injury.

In some embodiments the described methods may be carried out within about 72 hours of initiation of treatment for a subject's spinal cord injury. In some embodiments the described methods may be carried out within about 48 hours of initiation of treatment for a subject's spinal cord injury. In some embodiments the described methods may be carried out within about 24 hours of initiation of treatment for a subject's spinal cord injury. In some embodiments the described methods may be carried out within about 18 hours of initiation of treatment for a subject's spinal cord injury. In some embodiments the described methods may be carried out within about 12 hours of initiation of treatment for a subject's spinal cord injury. In some embodiments the described methods may be carried out within about 6 hours of initiation of treatment for a subject's spinal cord injury. In some embodiments the described methods may be carried out within about 4 hours of initiation of treatment for a subject's spinal cord injury. In some embodiments the described methods may be carried out within about 3 hours of initiation of treatment for a subject's spinal cord injury. In some embodiments the described methods may be carried out within about 2 hours of initiation of treatment for a subject's spinal cord injury. In some embodiments the described methods may be carried out within about 1 hour of initiation of treatment for a subject's spinal cord injury. In some embodiments the described methods may be carried out less than 1 hour after initiation of treatment for a subject's spinal cord injury.

Further disclosed herein are kits for producing a composition comprising one or more agents capable of modulating MCP-1 signaling and a biodegradable carrier, the kit comprising: one or more agents capable of modulating MCP-1 signaling; a biodegradable carrier; and instructions for producing said composition.

### EXAMPLES

### Microencapsulated TNF-α Inhibitor by Solvent Extraction/Evaporation, Single Oil-in-Water Emulsification

Biodegradable, polymeric microparticles were fabricated using a solvent extraction/evaporation, single oil-in-water (o/w) emulsification method. Carboxyl-terminated PLGA (0-20 wt %) and pirfenidone (0-20 wt %) were dissolved in a suitable, volatile organic solvent (e.g., dichloromethane, ethyl acetate). The resulting polymer solution dispersant phase was added to an aqueous continuous phase containing 0.5-5% (w/v) of surfactant (PVA) under constant shear rate mixing to create a single o/w microemulsion. The resulting stable microemulsion was subsequently added to an evaporation bath containing 200 mL of deionized water containing a trace concentration (0-0.5% (w/v)) of surfactant (PVA) under stirring at 350 rpm for 3 hours to effectively extract and evaporate the organic solvent. The hardened microparticles were then collected, purified with deionized water, and lyophilized.

Preparation of PLGA-g-PEG Nanoparticles and Subsequent Surface Bioconjugation of Anti-TNF-α Antibody Via Copper-Free Click Chemistry.

Varying ratios of PLGA-g-PEG and PLGA-g-PEG-azide diblock copolymer (0-1% by weight) are dissolved in a water miscible solvent (e.g. acetonitrile, dimethylsulfoxide, N,N-dimethylformamide, acetone). The polymer solution is precipitated into water, a nonsolvent, to yield nanoparticles comprising a PEGylated surface with varying percentages of PEG-azide functionality. The resulting nanoparticle suspension is stirred for 3-6 hours enable sufficient solvent diffusion. The nanoparticle suspension is then purified and concentrated by ultrafiltration and lyophilized. Azide-functional nanoparticles and dibenzylcyclooctyne-functionalized anti-TNF-α antibody (0.5-1 mole equivalent of terminal azide) are resuspended independently in buffered saline (pH 7.4) suspension and subsequently mixed for 30 minutes to covalently couple the antibody to the nanoparticle surface via copper-free click chemistry.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

What is claimed:

1. A composition for inhibiting inflammation in a subject with a spinal cord injury comprising:
one or more agents capable of specifically reducing TNF-α signaling; and
a biodegradable carrier.

2. The composition of claim 1, wherein the one or more agents comprise a TNF-α inhibitor, a protein that specifically binds TNF-α, an anti-inflammatory cytokine, or any combination thereof.

3. The composition of claim 2, wherein the protein that specifically binds TNF-α is etanercept, infliximab, adalimumab, certolizumab pegol, or any combination thereof.

4. The composition of claim 2, wherein the protein that specifically binds is an antibody.

5. The composition of claim 2, wherein the TNF-α inhibitor is pentoxifylline, methotrexate, pirfenidone, bupropion, or any combination thereof.

6. The composition of claim 2, wherein the anti-inflammatory cytokine is IL-10, IL-4, or any combination thereof.

7. The composition of claim 1, wherein the one or more agents are exposed on the surface of the biodegradable carrier, incorporated within the biodegradable carrier, or both.

8. The composition of claim 1, wherein the one or more agents are exposed on the surface of the biodegradable carrier.

9. The composition of claim 8, wherein the one or more agents comprise a protein that specifically binds TNF-α.

10. The composition of claim 1, wherein the one or more agents are incorporated within the biodegradable carrier.

11. The composition of claim 1, wherein the one or more agents are incorporated within the biodegradable carrier and exposed on the surface of the biodegradable carrier.

12. The composition of claim 11, wherein the one or more agents incorporated within the biodegradable carrier is an anti-inflammatory cytokine and the one or more agents exposed on the surface of the biodegradable carrier is a protein that specifically binds TNF-α.

13. The composition of claim 11, wherein the one or more agents incorporated within the biodegradable carrier is a TNF-α inhibitor, and the one or more agents exposed on the surface of the biodegradable carrier is a protein that specifically binds TNF-α.

14. The composition of claim 1, wherein the biodegradable carrier comprises a microparticle, a nanoparticle, a hydrogel, or any combination thereof.

15. The composition of claim 14, wherein the biodegradable carrier comprises PLGA, poly(ethylene glycol), a copolymer of PLGA and poly(ethylene glycol), or any combination thereof.

16. The composition of claim 14, wherein the microparticle is fabricated by emulsification.

17. The composition of claim 14, wherein the microparticle is fabricated by precipitation.

18. The composition of claim 14, wherein the microparticle is fabricated by spray drying.

19. The composition of claim 14, wherein the nanoparticle is fabricated by emulsification.

20. The composition of claim 14, wherein the nanoparticle is fabricated by nanoprecipitation.

21. The composition of claim 14, wherein the hydrogel is injectable and formed in situ.

22. The composition of claim 21, wherein the hydrogel is formed in situ by copper-free click chemistry crosslinking.

23. The composition of claim 21, wherein the hydrogel is formed in situ by reduced thiol/alkene Michael-type addition crosslinking.

24. The composition of claim 21, wherein the hydrogel is formed in situ by a shear thinning gelation mechanism.

25. The composition of claim 21, wherein the hydrogel is formed in situ by a thermosensitive gelation mechanism.

26. The composition of claim 1, wherein the biodegradable carrier degrades following administration to said subject.

27. The composition of claim 1, wherein the biodegradable carrier provides a therapeutically effective dose of the one or more agents for up to about 1 day, 2 days, 3 days, 4 days, 5 days, 6 days, 7 days, 8 days, 9 days, 10 days, 12 days, 14 days, 18 days, or 21 days.

28. The composition of claim 1, wherein the one or more agents reduces TNF-α signaling independent of modulating the cell cycle.

29. The composition of claim 1, further comprising a pharmaceutically acceptable carrier or excipient.

30. A method of treating inflammation in a subject having a spinal cord injury comprising administering to said subject the composition of claim 1.

31. The method of claim 1, wherein the composition is administered to the spinal cord of the subject.

32. The method of claim 1, wherein the composition is administered by direct injection into the spinal cord.

33. A kit for producing the composition of claim 1, the kit comprising:
a. one or more agents capable of specifically reducing TNF-α signaling;
b. a biodegradable carrier; and
c. instructions for producing said composition.

34. A composition for inhibiting inflammation in a subject with a spinal cord injury comprising:
one or more agents capable of modulating MCP-1 signaling; and
a biodegradable carrier.

35. The composition of claim 34, wherein the one or more agents is a JNK inhibitor, a TNF-α inhibitor, a protein that specifically binds TNF-α, a protein that specifically binds MCP-1, a COX inhibitor, a non-steroidal anti-inflammatory drug (NSAID), a COX-2 inhibitor, a tetracycline, an anti-inflammatory cytokine, methotrexate, pirfenidone, or any combination thereof.

36. The composition of claim 35, wherein the JNK inhibitor is SP600125.

37. The composition of claim 35, wherein the protein that specifically binds TNF-α is etanercept, infliximab, adalimumab, certolizumab pegol, or any combination thereof.

38. The composition of claim 35, wherein the protein that specifically binds MCP-1 is an antibody.

39. The composition of claim 38, wherein the antibody is ABN912.

40. The composition of claim 35, wherein the TNF-α inhibitor is pentoxifylline, methotrexate, pirfenidone, bupropion, or a mixture thereof.

41. The composition of claim 35, wherein the COX inhibitor is a NSAID.

42. The composition of claim 41, wherein the NSAID is ibuprofen or naproxen, or any combination thereof.

43. The composition of claim 35, wherein the COX-2 inhibitor is celecoxib, rofecoxib, curcumin, or any combination thereof.

44. The composition of claim 35, wherein the tetracycline is minocycline, doxycycline, or any combination thereof.

45. The composition of claim 35, wherein the anti-inflammatory cytokine is IL-10, IL-4, or any combination thereof.

46. The composition of claim 34, wherein one or more of said agents are exposed on the surface of the biodegradable carrier, incorporated within the biodegradable carrier, or both.

47. The composition of claim 34, wherein the one or more agents are exposed on the surface of the biodegradable carrier.

48. The composition of claim 47, wherein the one or more agents exposed on the surface of the biodegradable carrier comprise proteins that specifically bind TNF-α, proteins that specifically bind MCP-1, or both.

49. The composition of claim 34, wherein the one or more agents are incorporated within the biodegradable carrier.

50. The composition of claim 34, wherein the one or more agents are incorporated within the biodegradable carrier and exposed on the surface of the biodegradable carrier.

51. The composition of claim 50, wherein the one or more agents incorporated within the biodegradable carrier is an anti-inflammatory cytokine and the one or more agents exposed on the surface of the biodegradable carrier is a protein that specifically binds TNF-α.

52. The composition of claim 50, wherein the one or more agents incorporated within the biodegradable carrier is an anti-inflammatory cytokine and the one or more agents exposed on the surface of the biodegradable carrier is a protein that specifically binds MCP-1.

53. The composition of claim 50, wherein the one or more agents incorporated within the biodegradable carrier is a TNF-α inhibitor, a COX inhibitor, a COX-2 inhibitor, or a tetracycline and the one or more agents exposed on the surface of the biodegradable carrier is a protein that specifically binds TNF-α.

54. The composition of claim 50, wherein the one or more agents incorporated within the biodegradable carrier is a TNF-α inhibitor, a COX inhibitor, a COX-2 inhibitor, or a tetracycline and the one or more agents exposed on the surface of the biodegradable carrier is a protein that specifically binds MCP-1.

55. The composition of claim 34, wherein the biodegradable carrier comprises a microparticle, a nanoparticle, a hydrogel, or any combination thereof.

56. The composition of claim 55, wherein the biodegradable carrier comprises PLGA, poly(ethylene glycol), a copolymer of PLGA and poly(ethylene glycol), or any combination thereof.

57. The composition of claim 55, wherein the microparticle is fabricated by emulsification.

58. The composition of claim 55, wherein the microparticle is fabricated by precipitation.

59. The composition of claim 55, wherein the microparticle is fabricated by spray drying.

60. The composition of claim 55, wherein the nanoparticle is fabricated by emulsification.

61. The composition of claim 55, wherein the nanoparticle is fabricated by nanoprecipitation processing techniques.

62. The composition of claim 55, wherein the hydrogel is injectable and formed in situ.

63. The composition of claim 62, wherein the hydrogel is formed in situ by copper-free click chemistry crosslinking.

64. The composition of claim 62, wherein the hydrogel is formed in situ by reduced thiol/alkene Michael-type addition crosslinking.

65. The composition of claim 62, wherein the hydrogel is formed in situ by a shear thinning gelation mechanism.

66. The composition of claim 62, wherein the hydrogel is formed in situ by a thermosensitive gelation mechanism.

67. The composition of claim 34, wherein the biodegradable carrier degrades following administration to said subject.

68. The composition of claim 34, wherein the biodegradable carrier provides a therapeutically effective dose of the one or more agents for up to about 1 day, 2 days, 3 days, 4 days, 5 days, 6 days, 7 days, 8 days, 9 days, 10 days, 12 days, 14 days, 18 days, or 21 days.

69. The composition of claim 34, wherein the one or more agents modulate MCP-1 signaling independent of modulating the cell cycle.

70. The composition of claim 34, further comprising a pharmaceutically acceptable carrier or excipient.

71. A method of treating inflammation in a subject having a spinal cord injury comprising administering to said subject the composition of claim 34.

72. The method of claim 34, wherein the composition is administered to the spinal cord of the subject.

73. The method of claim 34, wherein the composition is administered by direct injection into the spinal cord.

74. A kit for producing the composition of claim 34, the kit comprising:
a. one or more agents capable of specifically reducing MCP-1 signaling;
b. a biodegradable carrier; and
c. instructions for producing said composition.
